(19) United States
(12) Patent Application Publication
(10) Pub. No.: US 2025/0381251 A1
(43) Pub. Date: Dec. 18, 2025

(54) Collagen and Cell Matter Supplement and Method for Administering Same

(71) Applicant: Lonza Greenwood LLC, Greenwood, SC (US)

(72) Inventors: Jim Lugo, Benicia, CA (US); Zainulabedin Saiyed, Benicia, CA (US)

(21) Appl. No.: 19/321,894

(22) Filed: Sep. 8, 2025

Related U.S. Application Data
(63) Continuation of application No. 17/260,360, filed on Jan. 14, 2021.
(60) Provisional application No. 62/698,384, filed on Jul. 16, 2018.

(51) Int. Cl. A61K 38/39; A23L 29/281; A23L 33/135; A61K 31/702; A61K 35/742; A61K 35/745; A61K 35/747; A61K 36/064

(52) U.S. Cl. CPC A61K 38/39; A23L 29/281; A23L 33/135; A61K 31/702; A61K 35/742; A61K 35/745; A61K 35/747; A61K 36/064

(57) ABSTRACT

The present disclosure is directed to a composition or nutritional supplement containing undenatured Type II collagen in combination with probiotic cell matter and/or a prebiotic. The different dietary or nutraceutical agents are capable of synergistically working together to improve the health and well-being of a human or animal. For instance, the nutritional supplement can be used to treat joint discomfort and pain, improve immune, or otherwise reducing inflammation in human and animal populations both at risk and actively presenting with such ailments.

### RELATED APPLICATIONS

The present application is based on and claims priority to U.S. Provisional Patent application Ser. No. 62/698,384, filed on Jul. 16, 2018, which is incorporated herein by reference.

### BACKGROUND

In recent years, the use of collagen to treat various conditions has become exceedingly popular. Collagen is a protein that can be found in muscles, bones, skin, blood vessels, and in other parts of the body. There are various types of collagen which differ in function and form. For instance, Type I collagen, the most abundant collagen, is made of fibers found in tendons, ligaments, organs, and skin. Type II collagen, on the other hand, primarily helps build cartilage, a major structural entity that sits on the surfaces of those bones which comprise articulating joints. Type III collagen, on the other hand, is made of fibers and is a major component of the extracellular matrix that makes up organs and skin. Type Ill collagen also forms blood vessels and tissue within the heart.

Numerous different products, including cosmetic creams and body lotions, contain collagen. Various oral supplements also contain collagen. Collagen production in the body, for instance, tends to slow as a person ages. For this reason, humans consume collagen supplements that reduce the effects of aging by improving the health of skin and hair.

Collagen has also been found to effectively treat arthritis and other joint health. For example, U.S. Pat. No. 9,066,926 discloses a method of reducing exercise-induced joint health in mammals by administering Type II collagen to a mammal. The '926 patent is incorporated herein by reference.

Various other supplements have been proposed containing bone material. For instance, U.S. Pat. No. 9,011,930 is directed to nutritional supplements containing demineralized bone matrix. The supplement may further contain a vitamin and is used to maintain or improve the condition of bones or cartilage.

In addition to treating joint health, collagen is known to treat leaky gut syndrome, a condition where toxins are able to pass through a digestive tract. Collagen intake can also boost metabolism, muscle mass, and energy output. Collagen can also improve liver health and improve cardiovascular health.

Although collagen can offer various advantages when administered to a human or animal, a need exists for a composition and method that can increase the effectiveness of collagen and/or work in conjunction with collagen to provide synergistic effects.

### SUMMARY

The present disclosure concerns a composition or nutritional product that includes a collagen source administered to a mammal, a patient, or a healthy individual in conjunction with a collagen adjuvant that augments the biological effects of the collagen. The collagen adjuvant, for instance, may comprise probiotic cell matter, a prebiotic, or a combination of probiotic cell matter and a prebiotic. The probiotic cell matter and/or prebiotic selected for use in the compositions, or products, that are the focus of the present disclosure are probiotic cell matter and/or prebiotics that are capable of reducing inflammation, such as the inflammation associated with joint health. For example, in one embodiment, the probiotic cell matter and/or prebiotic, when ingested by a mammal, induces production of anti-inflammatory cytokines in the body that synergistically operate with the collagen to provide health benefits to the mammal. For instance, in one embodiment, the collagen source in combination with the probiotic cell matter and/or prebiotic may work together to reduce joint health and/or repair surrounding joint tissue more quickly and/or more extensively (i.e., more effectively) than a single agent or class of agent alone can. The probiotic cell matter and/or prebiotic materials themselves can comprise either a single probiotic organism, probiotic component, or prebiotic molecule, a mixture of two or more probiotic organisms, probiotic components, two or more inactivated probiotic organisms (i.e., paraprobiotics), or prebiotic molecules, or any combination whatsoever of the above-mentioned embodiments.

In one embodiment, for instance, the present disclosure is directed to a nutritional product that includes a Type II collagen source plus probiotic cell matter, a Type II collagen source plus a prebiotic, or a Type II collagen source plus both probiotic cell matter and a prebiotic. In one embodiment, the Type II collagen source and the probiotic cell matter source and/or the prebiotic source are blended in a single composition or combined together and administered to the mammal. In an alternative embodiment, the Type II collagen source, the probiotic cell matter source, and the prebiotic source are delivered to a mammal separately, or are combined in various ways depending on the properties of the three source materials and class of molecules or organisms. The different components, for instance, can be combined in various ways using any suitable method, delivery platform, or technique that results in maximal bioavailability, stability or other desired properties.

In one embodiment, the Type II collagen source may comprise an undenatured, denatured, or hydrolyzed Type II collagen or a bioactive peptide derived from any of these Type II collagen sources. Probiotic cell matter and/or prebiotics that may be incorporated into the product include probiotic cell matter and prebiotics that modulate specific T cell types and/or modulate cytokine production such that an individual realizes improved joint health (e.g., flexibility & physical function) and/or a reduction in joint health.

In one embodiment, for instance, probiotic cell matter is used that comprises one or more probiotic organisms, paraprobiotics, as well as mixtures thereof including probiotic cell preparations. A probiotic organism may comprise, for instance, a bacteria or a eukaryotic organism, such as a yeast. For example, the probiotic organism may comprise a strain selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, and the Clostridias. Particular probiotic organisms that may be used include, but are not limited to, Escherichia coli, Bacteroides fragilis, various Bifidobacteria and Lactobacteria, including Lactobacteria casei, Bifidobacterium longum ssp infantis, Lactobacteria johnsonii, Lactobacteria rhamnosus, Lactobacteria reuteri, Lactobacteria acidophilus, Lactobacteria paracasei, Lactobacillus plantarum, and Lactobacillus lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof. In another embodiment, the probiotic organism may comprise, for instance, Saccharomyces cerevisiae, another member of the genus Saccharomycesceae, or other eukaryotic single cell organisms known or found to be a mammalian probiotics. In yet another embodiment, the probiotic organisms may comprise the cells of probiotic organisms that have been treated or altered but can be reconstituted, such as cells of probiotic organisms that have been lyophilized, or such as cells of probiotic organisms that have undergone heat treatment but remain viable.

As described above, in one embodiment, the probiotic cell matter may comprise a paraprobiotic (ghost probiotic) that constitutes the entire composition of the probiotic cell matter and can be combined with the collagen source. As used herein, a paraprobiotic comprises the killed, or inactivated cells of probiotic organisms, as well as cell fractions of probiotic organisms. For example, a paraprobiotic may comprise cells of probiotic organisms that have undergone heat treatment and are no longer viable, as well as cells of probiotic organisms that are inactivated, or killed, by changes in pH, by increased pressure, or by other means. For instance, in one aspect, the paraprobiotic includes lyophilized cells, heat-treated cells, or mixtures thereof, or in a futher aspect, includes one or more cell metabolites, cell hydrolysate, or cell extract thereof. In yet a further aspect, the paraprobiotic includes one or more yeast metabolites, where at least one nutrient is administered to the mammal in combination with the paraprobiotic. In another embodiment, the probiotic cell matter may comprise whole cells, crude cell fractions, or a milled cell mixture containing both intracellular and extracellular metabolites.

Probiotic cell matter also includes various different probiotic derivatives that may comprise mixtures of processed cell components of probiotic organisms, or mixtures of nutrients and one or more components derived from probiotic organism cells. For example, a probiotic derivative may comprise processed cell fractions that contain, for instance, metabolites, membranous components, or hydrolysates.

Alternatively, or in conjunction with the probiotic cell matter, the nutritional product or composition may contain a prebiotic. A prebiotic can be selected that increases the amount of a desired probiotic organism contained within the body, or is itself capable of inducing anti-inflammatory responses in the body. The prebiotic, for instance, may includearabinogalactans, arabinoxylans, alpha-glucan, beta-glucan, fructo-oligosaccharides, manno-oligosaccharides, galacto-oligosaccharides, xylo-oligosaccharides, mammalian milk oligosaccharides, extracellular vesicles, or mixtures thereof, as well as other molecules known to have prebiotic properties and/or known to activate the production of anti-inflammatory cytokines in the body.

Nonetheless, in one aspect, the Type II collagen source in conjunction with the probiotic cell matter, prebiotic, or both probiotic cell matter and prebiotic are administered to the mammal in an amount such that the mammal realizes a joint benefit. In yet a further aspect, the Type II collagen source in conjunction with the probiotic cell matter, prebiotic, or both probiotic cell matter and prebiotic, are administered to the mammal in amounts and ratios sufficient to modulate cytokine production or other immunoregulatory functions in a mammal. Additionally or alternatively, the Type II collagen source in conjunction with the probiotic cell matter, prebiotic, or both probiotic cell matter and prebiotic are administered to the mammal in amounts and ratios sufficient to modulate collagen production and/or joint repair in a mammal. Furthermore, in an aspect, the Type II collagen source in conjunction with the probiotic cell matter, the prebiotic, or both probiotic cell matter and prebiotic are administered to the mammal daily.

In one embodiment, the product can be a nutritional supplement in the form of individual dosage vessels. Each dosage vessel can be made from an orally acceptable composition. The dosage vessels, for instance, may comprise capsules, tablets, powders, or the like. Alternatively, the nutritional supplement may be in the form of a syrup or liquid suspension. Alternatively, the product of the present disclosure can be administered in a food or a beverage. For example, in one embodiment, the nutritional product may comprise a yogurt product. In an alternative embodiment, the nutritional product may comprise a fruit juice or other beverage.

When in the form of individual dosage vessels, each dosage vessel can contain the Type II collagen source in an amount from about 1 mg to about 15,000 mg, such as from about 1 mg to about 2,000 mg, such as from about 1 mg to about 1,000 mg, or such as from about 20 mg to about 100 mg. When present, the probiotic cell matter can be administered to a mammal in an amount from about 1 Million colony-forming units (CFUs) to about 200 Billion CFUs. An inactivated probiotic, or paraprobiotic, on the one hand, can be dosed in an amount from about 1 mg to about 10,000 mg. A prebiotic, on the other hand, can be administered to a mammal in an individual dose of an amount from about 1 mg to about 50,000 mg, or as a daily dose of from about 1 mg to about 50,000 mg.

In one embodiment, the present disclosure is also directed to a method for treating joint ailments, as described above, which are due to a disease, or age, (including arthritis), or to various normal strenuous activities (e.g., running, walking, stair climbing, exercising, etc.), in humans, at risk for joint health issues but who are free of disease, and other mammals. The method includes administering to the mammal a therapeutically effective amount of the various products or compositions described above. The compositions or products, which can be in the form of oral dosage vessels, can be administered to the mammals in an amount sufficient to reduce the joint discomfort or pain, or to begin and support the healing process, or any combination thereof.

In a further aspect, the present disclosure may generally be directed to a nutritional product that includes a Type II collagen source and a collagen adjuvant, where the collagen adjuvant includes probiotic cell matter, a prebiotic, or mixtures thereof. In one aspect, the collagen adjuvant in the nutritional product includes probiotic cell matter, such as a probiotic organism, a paraprobioitc, or mixtures thereof. Additionally or alternatively, the probiotic cell matter is a probiotic organism, where the probiotic organism includes a bacterial or a eukaryotic organism. In one aspect, the probiotic organism is a strain selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, and the Clostridias, and, in yet a further aspect, the probiotic organism is Escherichia Coli, Bacteroides fragilis, various Bifidobacteria and Lactobacteria, including Lactobacteria casei, Bifidobacterium longum ssp infantis Lactobacteria johnsonii, Lactobacteria rhamnosus, Lactobacteria reuteri, Lactobacteria acidophilus, Lactobacteria paracasei, Lactobacillus plantarum, and Lactobacillus Lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof. Furthermore, in an aspect, the probiotic organism is an organism from the genus Saccharomycesceae such as Saccharomyces cerevisiae.

Moreover, in another aspect, the probiotic cell matter is a paraprobioitc. In one aspect, in the paraprobiotic includes lyophilized cells, heat-treated cells, a cell hydrolysate, a cell extract, a cell metabolite, or mixtures thereof. Additionally or alternatively, the paraprobiotic is a yeast metabolite or mixture of metabolites optionally combined with a nutrient.

In yet a further aspect, the collagen source and the collagen adjuvant are blended together in a nutritional product. In one aspect, the collagen source and the collagen adjuvant are not blended together, but are provided as separate entities in the nutritional product. Furthermore, in an aspect, the product is a nutritional supplement for taking orally. Moreover, in one aspect, the Type II collagen source is an undenatured, denatured, or hydrolyzed Type II collagen or bioactive peptide derived from Type II collagen.

The present disclosure is also directed to various methods to improve the health of a user in addition to reducing joint discomfort or pain. For instance, in one embodiment, the composition of the present disclosure can be used to improve immune system health and/or decrease inflammation.

### DEFINITIONS

The term “therapeutically effective amount” as used herein, with respect to the composition described above, shall mean that dosage, or amount of composition, that provides the specific pharmacological or nutritional response for which the composition (containing the collagen source combined with probiotic cell matter and/or prebiotic) is administered or delivered to subjects in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “delivering” or “administering” as used herein, refers to any route for providing the composition, product, or a nutraceutical, to a subject as accepted as standard by the medical community. For example, the present invention contemplates routes of delivering or administering that include oral ingestion plus any other suitable route of delivery including transdermal, intravenous, intraperitoneal, intramuscular, topical and subcutaneous.

The term “encapsulate”, “encapsulated”, or “encapsulating” refers to any compound that is completely surrounded by a protective material. For example, a compound may become encapsulated by a population of nanoemulsion particle formation during microfluidization.

The term “nutraceutical” and “ingredient” refers to any compound added to a dietary source (e.g., a fortified food or a dietary supplement) that provides health or medical benefits in addition to its basic nutritional value.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to products or compositions, such as nutritional supplements, medicinal and/or food formulations, or beverages for administering to humans and animals that can be used as a daily supplement or to treat a particular ailment. Of particular advantage is that the composition can be formulated to provide multiple health benefits simultaneously.

In one embodiment, for instance, the composition of the present disclosure is particularly formulated to improve joint health. For instance, the product or composition can be used to treat arthritis or non-arthritic joint health, joint discomfort, or lack of joint flexibility. The composition of the present disclosure can be used also to improve the composition of the microbiome in mammals.

In general, the composition of the present disclosure contains a collagen, such as denatured or undenatured Type II collagen, in combination with an adjuvant that, in one embodiment, can work synergistically with the collagen. For example, in one embodiment, the adjuvant can increase the effectiveness of the collagen.It is also believed that using the combination collagen, in particular undenatured collagen in combination with the probiotic can reduce the time in which the undenatured collagen can effectively reduce joint pain and/or improve joint function. This may be also referred to a quick on-set of action.

In one embodiment, for instance, Type II collagen is administered in combination with probiotic cell matter, a prebiotic, or both probiotic cell matter and a prebiotic. The probiotic cell matter may contain a probiotic such as one or more viable organisms, while the prebiotic may be comprised of one or more distinct prebiotic fiber types. The probiotic cell matter and/or prebiotic may act as an adjuvant for the Type II collagen source. For instance, the probiotic cell matter and/or prebiotic may increase the percentage of mammals that favorably respond to being administered a Type II collagen source. The probiotic cell matter and/or prebiotic may accelerate the onset of action attributed to taking routine dosage of Type II collagen in mammals. For example, in one embodiment, the probiotic cell matter and/or prebiotic may prime the immune system of the mammal that synergistically operates in conjunction with the Type II collagen source for providing faster and/or more enhanced treatment to the patient.

Probiotic cell matter includes probiotic organisms, which include viable bacteria and yeast cells; and paraprobiotics, which include the killed or inactivated cells of probiotic organisms and/or the crude cell fractions of probiotic organisms including probiotic derivatives, which include the processed cell components of probiotic organisms. Probiotic cell matter provides health benefits when consumed or administered to a mammal. Probiotic cell matter, for instance, can improve immune system reaction, reduce inflammation, and/or reduce gastrointestinal discomfort in mammals. In one embodiment, the probiotic cell matter selected for use in the present disclosure is probiotic cell matter that increases the presence of T cell types in the body and/or increases the production of cytokines in the body. For example, probiotic cell matter may be selected that induces T regulators (such as CD4⁺, CD25⁺, Foxp3⁺ T regulator cells) to modulate or suppress specific immune responses and thereby increase the production of anti-inflammatory cytokines, such as transforming growth factor beta (TGF-β) or interleukin-10 (IL-10), which can enhance the effectiveness of the Type II collagen source.

Probiotic cell matter, for instance, can modulate immune system responses in mammals either directly or indirectly. Probiotic cell matter can indirectly affect the immune system by maintaining or repairing epithelial barriers or by increasing the production of fatty acids, such as short chain fatty acids that have anti-inflammatory properties, through a number of distinct mechanisms.

Probiotic cell matter can also have a direct effect on immune system responses. For instance, probiotic cell matter can beneficially influence the differentiation and function of a number of immune cell types associated with an inflammatory response. For instance, probiotic cell matter can have an influence on dendritic cells, Natural Killer (NK) cells, as well as T cells. These cells can increase the production of cytokines in the body that help promote the differentiation of T cells into various different types of regulatory cells that can stimulate immune responses. In addition, probiotic cell matter can influence the synthesis of regulatory cytokines, such as IL-10 and TGF-β, which exert inflammation-modulating effects on the body and can not only reduce inflammation but also work synergistically with a collagen source to alleviate pain or discomfort and provide healing effects. This influence can occur via stimulation of the innate and adaptive immune systems, as stated above, or via epigenetic phenomena that modulate the gene expression of anti-inflammatory and inflammatory mediators. It may also occur via the direct impact of these cytokines on the structure function of nocioceptive pain receptors.

Probiotic organisms that may be used in the accordance with the present disclosure include various beneficial bacteria and yeast. For instance, a probiotic organism may comprise gram-positive bacteria, gram-negative bacteria, or various different eukaryotic cells, such as yeast.

In one embodiment, for instance, the probiotic organism used in accordance with the present disclosure comprises a type of bacteria. The bacteria may be selected from various different phyla including strains selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, and the Clostridias. Specific examples of probiotics that may be used include, but are not limited to, Escherichia coli, Bacteroides fragilis, various Bifidobacteria and Lactobacteria, including Lactobacteria casei, Bifidobacterium longum ssp infantis, Lactobacteria johnsonii, Lactobacteria rhamnosus, Lactobacteria reuteri, Lactobacteria acidophilus, Lactobacteria paracasei, Lactobacillus plantarum, and Lactobacillus lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof.

In an alternative embodiment, eukaryotic cells may be used as the probiotic organism. For instance, the probiotic organism may comprise Saccharomyces cerevisiae.

In another alternative embodiment, the probiotic organism may comprise bacteria or yeast cells that have been treated or altered but remain viable. For instance, probiotic organism cells that have been lyophilized, or freeze-dried, but can be reconstituted, or probiotic organism cells that have undergone heat treatment, but retain viability, may be used in accordance with the present disclosure.

Paraprobiotics that may be used in accordance with the present disclosure include the killed or inactivated cells of probiotic organisms and the cell fractions of probiotic organisms. In one embodiment, the paraprobiotic may comprise bacteria or yeast cells that have undergone heat treatment and are no longer viable but are still able to provide health benefits when consumed or administered to a mammal. Alternatively, the paraprobiotic may comprise viable or nonviable cells possibly subjected to changes in pH, increased pressure, milling, or the like, and in various combinations.

In yet another embodiment, the paraprobiotic may comprise crude cell fractions containing, for instance, metabolites or hydrolysates. For example, protein hydrolysates extracted from yeast may be used in accordance with the present disclosure.

Other paraprobiotics that may be used in accordance with the present disclosure include probiotic derivatives that may include processed cell components of probiotic organisms, mixtures of processed probiotic organism cell components, and mixtures of nutrients and one or more components derived from probiotic organism cells. In one embodiment, the probiotic derivative may comprise processed cell fractions which contain, for instance, metabolites or hydrolysates. In another embodiment, a mixture made up of beneficial nutrients and metabolites that are produced by yeast, such as the EpiCor® brand fermentate, which is commercially available from Embria Health Sciences, LLC, may be used in accordance with the present disclosure.

In addition to probiotic cell matter, or instead of probiotic cell matter, the product or composition of the present disclosure may contain a prebiotic. Prebiotics are non-digestible food ingredients that can either stimulate or support the growth of desired probiotics and thereby indirectly improve the joint health efficacy of Type II collagen supplements, or can improve the joint health efficacy of Type II collagen supplements directly. Prebiotics, for instance, can be used to accelerate or otherwise enhance the growth of a probiotic contained within the body. Alternatively, a prebiotic can be administered to a mammal in conjunction with a probiotic to maintain optimum growth conditions for the probiotic within the body, or to induce the probiotic to produce metabolites beneficial to the host. Prebiotic molecules may also stimulate the immune system directly via the specific binding to receptors that recognize the complex, repeating nature of prebiotic molecules. Examples of prebiotics that may be used include arabinogalactans, alpha glucan, beta glucan, fructo-oligosaccharides, mammalian milk oligosaccharides of various types and molecular weights, extracellular vesicles, or mixtures thereof. This listing of prebiotics is not intended to be all-inclusive.

In addition to probiotic cell matter and/or a prebiotic, the product or composition of the present disclosure also contains a collagen source, particularly a Type II collagen, such as an undenatured Type II collagen. Type II collagen for use in the present disclosure can be obtained from any suitable source. For instance, the collagen can be derived from a variety of mammalian sources, avian sources, or fish species. For instance, the collagen can be obtained from salmon, shark, poultry, eggshells, turkey cartilage, bovine cartilage, and the like. In one embodiment, for instance, the Type II collagen can be obtained as disclosed in U.S. Pat. No. 7,083,820 to Schilling, which is incorporated by reference. For example, undenatured Type II collagen is available commercially as the UC-II® brand from InterHealth Nutraceuticals. The UC-II® brand is a natural ingredient that contains a glycosylated, undenatured Type II collagen derived from chicken sternum. The collagen source can also comprise a hydrolyzed collagen as well as a pure native collagen protein that can be produced via enzyme hydrolysis, or fermentation techniques, or other methods known to those skilled in the art. In one embodiment, the collagen source can be free of any bone or bone material. In other embodiments, the collagen source can be free of any TGF-B, bone morphogenic proteins, or both. In still another embodiment, the collagen source comprises Type II collagen.

In preparing animal tissue for oral administration, in one embodiment, the undenatured Type II collagen-containing tissue can be first dissected free from surrounding tissues and diced or otherwise separated into particles. The particulate, or milled, cartilage can then be sterilized by means that do not affect or denature the structure of most of the Type II collagen in the tissue. This material is finally formed into doses containing therapeutically effective levels of undenatured Type II collagen, said levels being generally in the amount of at least about 0.001 gram and preferably from about 0.001 grams to about 0.5 grams of animal tissue per dose. Because this is a natural product, some variation from sample to sample is to be expected. These variations can be minimized by blending after separation into particles. The blending is aided by analytical techniques that allow the amount of undenatured Type II collagen and other constituents to be measured.

These measurements will allow batches to be blended for uniformity.

Hydrolyzed collagen comes from skin, bone, or connective tissue, a known byproduct of the meat industry (gelatin). Breakdown of the type II collagen (e.g., joints) to small peptides is achieved via enzymatic, chemical, or a mix of both of these procedures. This material is dosed at about 0.001 gram and preferably from about 0.01 gram to about 15 grams of hydrolyzed powder per daily dose.

A nutritional product or composition of the present disclosure can be administered in an oral form. The collagen source and the probiotic cell matter and/or prebiotic may be administered to a mammal separately or can be administered together, in part or in whole. In one embodiment, for instance, the collagen source can be mixed together with the probiotic cell matter and/or the prebiotic.

In one embodiment, the nutritional product is formed into a dosage vessel, such as a tablet or capsule that can be taken orally. The nutritional product, for instance, can be manufactured in the form of capsules, tablets, gummy chewables, edible films, lozenges, powders, liquid suspensions, syrups, and the like. The collagen source and the probiotic cell matter and/or the prebiotic may be combined together within the dosage vessel or can be contained in the dosage vessel and separated from one another. Alternatively, the components of the nutritional product can be administered separately, each of which components can exist in alternative, different forms.

Alternatively, the collagen source, the probiotic cell matter, and/or the prebiotic alone or in combination can be contained in a food product or in a beverage. For instance, the nutritional product may comprise a yogurt. Alternatively, the nutritional product may comprise a beverage, such as a soft drink, milkshake, beer, or even coffee.

The amount of Type II collagen and the amount of the probiotic cell matter and/or prebiotic in a dose consumed at any given time will vary with the purpose of consumption and the severity of symptoms, as well as the condition, age, weight, medical history, and general physical characteristics of the subject (human or animal) to be treated or supplemented. Consequently, the dosages, frequency, and period of time over which dosages are administered can vary widely. The nutritional product of the present disclosure can be combined with other digestible ingredients, such as those in the form of aqueous dispersions such as milk, or with other protein-rich substances, sugars, and starches. In one embodiment, the nutritional product may be administered directly as a comminuted solid as in an encapsulated comminuted solid, such as a compression and formed pill, as well as a slurry with or without other digestible compositions such as, for example, foodstuffs.

In one embodiment, the ratio between the Type II collagen and the probiotic cell matter can generally be from about 1 mg:200 Billion CFUs to about 1 mg:1 Million CFUs, such as from about 40 mg:200 Billion CFUs to about 40 mg:1 Million CFUs, such as from about 80 mg:200 Billion CFUs to about 80 mg:1 Million CFUs, such as from 320 mg:200 Billion CFUs to about 320 mg:1 Million CFUs, and all such combinations up to and including 15,000 mg:200 Billion CFUs to 15,000 mg:1 Million CFUs, including all possible combinations and ratios covered by the above stated high and low dosages for the Type II collagen and the probiotic cell matter.

In one embodiment, the ratio between the Type II collagen and the paraprobiotic can generally be from about 1 mg:1 mg to about 1 mg:10,000 mg, such as from about 40 mg:1 mg to about 40 mg:10,000 mg, such as from about 80 mg:1 mg to about 80 mg:10,000 mg, such as from 320 mg:1 mg to about 320 mg:10,000 mg, and all such combinations up to and including 15,000 mg:1 mg to 15,000 mg:10,000 mg, and includes all possible combinations and ratios covered by the above stated high and low dosages for the Type II collagen and the paraprobiotic.

In one embodiment, the ratio between the Type II collagen and the prebiotic can generally be from about 1 mg:1 mg to about 1 mg:50,000 mg, such as from about 40 mg:0.001 g to about 40 mg:50,000 mg, such as from about 80 mg:1 mg to about 80 mg:50,000 mg, such as from 320 mg:1 mg to about 320 mg:50,000 mg, and all such combinations up to and including 15,000 mg:1 mg to 15,000 mg:50,000 mg, and includes all possible combinations and ratios covered by the above stated high and low dosages for the Type II collagen and the prebiotic.

In one embodiment, the composition or nutritional product of the present disclosure is formed into individual dosage vessels that are intended to be taken orally by a human or animal. The dosage vessel, for instance, may comprise a tablet or capsule. In one embodiment, the collagen source and the probiotic cell matter and/or prebiotic may be contained in a single dosage vessel. Alternatively, only one of the above ingredients may be contained in the dosage vessel while the other ingredient(s) are contained in a separate dosage vessel or in a different delivery device.

In other embodiments, the nutritional product can be created using any dose of the Type II collagen with any dose of the probiotic cell matter or prebiotic, in any of the delivery platforms described above.

In one embodiment, the Type II collagen can be present in each dosage vessel generally in an amount from about 1 milligram to about 15,000 milligrams. The amount of Type II collagen contained in a dosage vessel can depend upon the type of delivery platform. For instance, the Type II collagen can be present at any of the above amounts and at any increment when contained in a food or beverage product. When contained in a capsule or pill, however, each dosage vessel may contain the Type II collagen in an amount generally from about 1 milligram to less than about 5,000 milligrams, such as less than about 2,000 milligrams, such as less than about 1,000 milligrams.

When denatured or hydrolyzed collagen is present in the composition, the collagen can be present in each dosage vessel in an amount from about 500 milligrams to about 15,000 milligrams. When undenatured Type II collagen is used to produce the composition, on the other hand, each dosage vessel can contain the collagen in an amount from about 1 milligram to about 5,000 milligrams. For instance, the Type II collagen can be present in each dosage vessel in an amount greater than about 5 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The amount of Type II collagen present in each dosage vessel can generally be less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams.

Each dosage vessel as described above can be taken periodically depending upon the condition of the user and various characteristics of the user. For instance, each dosage vessel can be taken daily, can be taken every two days, can be taken every three days, can be taken every four days, can be taken weekly, or the like. In addition, it should be understood that each dosage vessel may be taken more than once daily. For instance, in certain circumstances, from about two to about four dosage vessels may be taken each day.

As described above, in one embodiment, the composition of the present disclosure can be formulated as a nutritional supplement or product that is taken orally. It should be understood, however, that the composition or product of the present disclosure can be administered to a human or animal using any suitable method. In one embodiment, the collagen source can be incorporated into a topical composition that is intended to be applied to the skin of a user while the probiotic cell matter or prebiotic is administered orally. When formulated as a topical composition, for instance, the collagen source can be blended, or separately delivered, with various ingredients and components. For instance, when formulated as a topical composition, the collagen source can be blended with solvents, surfactants, emulsifiers, consistency factors, conditioners, emollients, skin care ingredients, moisturizers, thickeners, lubricants, preservatives, and various different dermatological ingredients. When applied to the skin of a user, the collagen source can be used to improve skin health and/or can be formulated so as to be absorbed into the body.

In addition to being directed to a composition or a nutritional product, the present disclosure is also directed to a method for administering the nutritional product to a human or animal. For example, in one embodiment, the present disclosure is directed to a method for maintaining joint health and/or treating joint health including arthritis by administering to a mammal a nutritional product containing undenatured Type II collagen in combination with probiotic cell matter and/or a prebiotic. The nutritional supplement is administered to the mammal in a therapeutically effective amount so as to maintain joint health and/or reduce joint health or to otherwise treat join pain. In one embodiment, for instance, the reduction in joint health is evidenced by improvements in range of motion, increased levels of activity, improved knee flexibility, or any activity that depends on having functional knees. In general, as described above, the composition of the present disclosure can be administered orally. In other embodiments, however, the composition can be administered subcutaneously or via a patch.

The following examples illustrate the invention without limitation. All parts and percentages are given by weight unless otherwise indicated.

Furthermore, certain embodiments of the present disclosure may be better understood according to the following example, which is intended to be non-limiting and exemplary in nature. It will be understood that each of the elements described in the examples below, or two or more together may also find a useful application in other types of methods differing from the type described above.

### EXAMPLE

A single intra-articular injection of sodium monoiodoacetate (MIA) will be introduced into the knee joint of rats according to the method described by Fernihough J et al., 2004. Sodium monoiodacetate (MIA) inhibits chondrocyte metabolism leading to cartilage degradation in form of osteoarthritic-like focal lesions in the cartilage associated with subchondral bone thickening 14 days after administration (Guingamp et al., 1997). This model therefore can easily and quickly reproduce osteoarthritic-like lesions and functional impairment in rats, similar to that observed in human disease (Guzman et al., 2003). After 7 days post-injection, the inflammatory component subsides and the remaining pain is considered neuropathic in nature. Briefly, rats were deeply anaesthetized. Following abolition of the hind paw pinch withdrawal reflex, a needle was introduced into the joint cavity between the tibial plateau and femoral condyles.

Once in place, 2 mg of MIA in a carrier will be injected into one knee joint and the rat was allowed to recover for 14 days prior to pain assessment.

The rats will receive a daily administration of the formulations shown in Table 1 below based on the weight of each rat. The rate of application is shown in Table 1The undenatured Type-Il collagen was UC-II® brand available from Lonza, having offices in Morristown, NJ. The probiotic is EpiCor® which is commercially available from Embria Health Sciences, LLC,

TABLE 1

| | Amount of undenatured | | |
|---|---|---|---|
| Example | Type-II collagen | Probiotic | |
| A | 0.66 mg/kg/day | 4.125 | mg/kg/day |
| B | 0.66 mg/kg/day | 8.25 | mg/kg/day |
| C | 0.66 mg/kg/day | 16.5 | mg/kg/day |
| D | 0.66 mg/kg/day | 0 | |
| E | 0 | 4.125 | mg/kg/day |
| F | 0 | 8.25 | mg/kg/day |
| G | 0 | 16.5 | mg/kg/day |

Each of the above Example formulations tested in rates treated above to determine the effect of the combinations of ingredients affect the joint function of the knee joint of each rat with respect to joint pain, joint function and range of motion of the knee joint.

In other embodiments, the present disclosure is directed to a method of treating a mammal in order to improve immune health or reduce inflammation. The method includes administering to the mammal the nutritional supplement as described above in a therapeutically effective amount sufficient to have one of the above effects.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1-43. (canceled)

44. A method of modulating joint health in a mammal comprising:
administering to the mammal a therapeutically effective amount of a nutritional supplement, wherein the nutritional supplement comprises:
a Type II collagen source, wherein the Type II collagen source comprises an undenatured, denatured, or hydrolyzed Type II collagen or bioactive peptide derived from Type II collagen; and
a collagen adjuvant, the collagen adjuvant comprising probiotic cell matter, a prebiotic, or mixtures thereof,
wherein a ratio between the Type II collagen source and the probiotic cell matter, when present, is from 1 mg:3 mg to 1 mg:50 mg, and a ratio between the Type II collagen and the prebiotic, when present, is from 1 mg:1 mg to 1 mg:50,000 mg.

45. The method of claim 44, wherein the collagen adjuvant comprises the probiotic cell matter, the probiotic cell matter comprising a probiotic organism, a paraprobiotic, or mixtures thereof.

46. The method of claim 45, wherein the probiotic cell matter comprises the probiotic organism, the probiotic organism comprising a bacteria or a eukaryotic organism.

47. The method of claim 46, wherein the probiotic organism comprises a strain selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, and the Clostridias, or comprises Escherichia coli, Bacteroides fragilis, Bifidobacteria and Lactobacteria, including Lactobacteria casei, Bifidobacterium longum ssp infantis, Lactobacteria johnsonii, Lactobacteria rhamnosus, Lactobacteria reuteri, Lactobacteria acidophilus, Lactobacteria paracasei, Lactobacillus plantarum, and Lactobacillus lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof.

48. The method of claim 46, wherein the probiotic organism comprises an organism from the genus Saccharomycesceae such as Saccharomyces cerevisiae.

49. The method of claim 45, wherein the probiotic cell matter comprises a paraprobiotic, wherein in the paraprobiotic comprises lyophilized cells, heat-treated cells, a cell hydrolysate, a cell extract, a cell metabolite, or mixtures thereof.

50. The method of claim 45, wherein the probiotic cell matter comprises a paraprobiotic, wherein the paraprobiotic comprises a yeast metabolite or mixture of metabolites optionally combined with a nutrient.

51. The method of claim 44, wherein the Type II collagen source and the collagen adjuvant have been blended together or are provided as separate entities.

52. The method of claim 44, wherein the nutritional supplement is administered orally.

53. The method of claim 44, wherein the collagen adjuvant comprises the prebiotic and wherein the prebiotic comprises arabinogalactans, arabinoxylans, alpha-glucan, beta-glucan, fructo-oligosaccharides, manno-oligosaccharides, galacto-oligosaccharides, xylo-oligosaccharides, mammalian milk oligosaccharides, extracellular vesicles, or mixtures thereof.

54. The method of claim 44, wherein the nutritional supplement comprises a food product or a beverage.

55. The method of claim 54, wherein the nutritional supplement comprises tablets, capsules, gummy chewables, edible films, liquid suspensions, powders, syrups, or lozenges, a yogurt, a fruit juice or other beverage matrix.

56. The method of claim 44, wherein the Type II collagen source comprises an undenatured Type II collagen.
